(19) United States
(12) Patent Application Publication
(10) Pub. No.: US 2021/0214788 A1
(43) Pub. Date: Jul. 15, 2021

(54) METHOD FOR DETECTING, LOCATING AND MONITORING SEEPAGE AND LEAKAGE OF HYDRAULIC STRUCTURES

(71) Applicant: HUNAN ZONSEN PEPLIB BIOTECH CO., LTD, Zhuzhou, Hunan (CN)

(72) Inventors: Zhuying WANG, Monmouth Junction, NJ (US); Mingxia SHEN, Nanjing (CN); Linhua JIANG, Nanjing (CN)

(21) Appl. No.: 16/072,493

(22) PCT Filed: Jan. 26, 2016
(86) PCT No.: PCT/CN2016/072167

§ 371 Date: Jul. 24, 2018

(51) Int. Cl. C12Q 1/6876

(52) U.S. Cl. CPC C12Q 1/6876

(57) ABSTRACT

The disclosure relates to an improved method for detecting, locating and monitoring fluid seepage and leakage from a hydraulic work with superior sensitivity. The method includes using a DNA sequence as the probe to trace the fluid seepage and leakage from a hydraulic work. The probe can be captured and then amplified more than a millionfold by an enzymatic method such as the polymerase chain reaction (PCR) to give a high detection signal. Even a single molecule of the DNA probe can be detected by an enzymatic amplification, thus to give superior sensitivity. The improved detection method is applicable to detecting, locating and monitoring fluid seepage and leakage from hydraulic works, the improved method can also be used, for example, to trace the groundwater flow, underground water flow and other liquid flow. Other related methods are also described.

### FIELD OF THE INVENTION

The invention relates to an improved method for detecting, locating and monitoring fluid seepage and leakage from a hydraulic work with superior sensitivity. The method includes using a DNA sequence as the probe to trace the fluid seepage and leakage from a hydraulic work. The probe can be captured and then amplified more than a millionfold by an enzymatic method such as the polymerase chain reaction (PCR) to give a high detection signal. Even a single molecule of the DNA probe can be detected by an enzymatic amplification, thus to give superior sensitivity. The improved detection method is applicable to detecting, locating and monitoring fluid seepage and leakage from hydraulic works, the improved method can also be used, for example, to trace the groundwater flow, underground water flow and other liquid flow.

### BACKGROUND OF THE INVENTION

Hydraulic works such as dams and reservoirs are an essential asset of great benefit to modern society and play important roles in the development of human society (Environment Agency. Post-incident reporting for UK dams. 2007. Annual Report). Some important uses of dams and reservoirs include water supply, hydropower production, irrigation, drainage and flood control, etc. (Amanda Briney. Overview of Dams and Reservoirs, http://geography.about.com/od/waterandice/a/damsreservoirs.htm). However, they can also be massively destructive and potentially cause great damage and loss of life. One of the major causes of catastrophic failure is related to uncontrolled water seepage and piping from the dams and reservoirs, which threaten dam stability (A I H Malkawi, M Al-Sheriadeh. Evaluation and rehabilitation of dam seepage problems. A case study: Kafrein dam. Engineering Geology. 2000, 56(s 3-4):335-345). Therefore, it is very important to detect seepage and leakage from a hydraulic work at very early stage to prevent the deterioration, hence to avoid possible catastrophic dam failure. Historically, many substances such as salts, particles, dyes and fluorescent dyes, etc. have been used as tracers to trace the water paths or detect seepage of dams, however, these tracers have a common disadvantage of being not sensitive enough, and usually a large quantity of the tracer is needed.

Radioactive isotopes were later used as tracers because their radioactivity is easy to detect, and relatively much less radioactive material is needed since the radiation emitted is so easy to detect (Uses of Radioactive Isotopes section 11.4 from the book “Introduction to Chemistry: General, Organic, and Biological (v. 1.0)”). Radioactive tracers were successfully used to determine the location of fractures created by hydraulic fracturing in natural gas production (Reis, John C. Environmental Control in Petroleum Engineering. 1976. Gulf Professional Publishers).

Although radioactive isotopes are now being commonly used as effective tracers in many different fields, there are some disadvantages related to the use of radioactive isotopes. Some of the disadvantages include safety hazards, generation of radioactive waste, toxicity to organisms, and radioactive decay leading to loss of signal over time, etc. The cost related to the production, transportation, usage and disposal of radioactive isotopes is also an issue.

Thus, there is still a need for a safer, more cost-effective and more sensitive tracing method for detecting, locating and monitoring fluid seepage and leakage from a hydraulic work. Embodiments of the present invention relate to such a method for detecting, locating and monitoring fluid seepage and leakage from a hydraulic work more sensitively and safely.

### BRIEF SUMMARY OF THE INVENTION

It is an object of this invention to provide novel methods for tracing the flow of liquids with superior sensitivity using nucleic acids of specific sequences as tracers. It is an object of this invention to provide a method for detecting, locating and monitoring fluid seepage and leakage from a hydraulic work with superior sensitivity. The method comprising: (i) designing a specific DNA sequence with a specific length; (ii) producing and using the nucleic acid containing the DNA sequence as the probe and applying the probe to a proper location of the hydraulic work; (iii) taking samples from specific locations that may contain the probe; (iv) amplifying the probe in the samples by an enzymatic amplification method; and (v) determining the amount or copy number of the probe in the samples to analyze fluid seepage and leakage from the hydraulic work.

It is another object of this invention to provide a method for tracing the flow of the groundwater or underground water with superior sensitivity. The method comprising: (i) designing a specific DNA sequence with a specific length; (ii) producing and using the nucleic acid containing the DNA sequence as the probe and applying the probe to a proper location of the groundwater or underground water; (iii) taking samples from specific locations that may contain the probe; (iv) amplifying the probe in the samples by an enzymatic amplification method; and (v) determining the amount or copy number of the probe in the samples to analyze the flow of the groundwater or underground water.

It is yet another object of this invention to provide a method for tracing the flow of liquids with superior sensitivity. The method comprising: (i) designing a specific DNA sequence with a specific length; (ii) producing and using the nucleic acid containing the DNA sequence as the probe and applying the probe to a proper location of the liquid body; (iii) taking samples from specific locations that may contain the probe; (iv) amplifying the probe in the samples by an enzymatic amplification method; and (v) determining the amount or copy number of the probe in the samples to analyze the flow of the liquid.

It is yet another object of this invention to provide a method for efficiently tracing the flow of liquids with superior sensitivity. The method comprising: (i) designing multiple specific DNA sequences with specific lengths; (ii) producing and using the nucleic acids containing the DNA sequences as the probes and applying the probes to different locations of the liquid body; (iii) taking samples from specific locations that may contain the probes; (iv) amplifying the probes in the samples by an enzymatic amplification method; and (v) determining the amount or copy numbers of the probes in the samples to analyze the flow of the liquid.

It is a further object of this invention to provide an alternative method for tracing the liquid flow with superior sensitivity using nucleic acids of specific sequences as tracers. Unlike other tracers, many atoms or molecules are needed to be present for the tracer to be detected, for DNA tracers, a single molecule of DNA sequence can be efficiently amplified by an enzymatic amplification method to more than a millionfold and then easily detected. Thus, when DNA molecules are used as a tracer, superior sensitivity can be reached. In other words, single-molecule sensitivity can be realized when DNA molecules are used as a tracer, which will significantly reduce the amount of a tracer to be used. Another advantage for this method is that multiple DNA sequences of different sizes can be used simultaneously to further increase the tracing efficiency.

Additional objects of the invention are reflected in the original claims. The details of embodiments of the disclosure are set forth in the accompanying drawings and the description below. Other features, objects, and advantages will be apparent from the description and drawings, and from the claims.

### BRIEF DESCRIPTION OF THE SEVERAL VIEWS OF THE DRAWINGS

The foregoing brief summary, as well as the following detailed description of the invention, will be better understood when read in conjunction with the appended drawings. For the purpose of illustrating the invention, there are shown in the drawings embodiments which are presently preferred. It should be understood, however, that the invention is not limited by the drawings presented.

In the drawings:

FIG. 1 schematically illustrates the DNA sequence of a DNA tracer according to an embodiment of the invention;

FIG. 2 schematically illustrates a DNA vector containing the DNA sequence of a DNA tracer;

FIG. 3 schematically illustrates a PCR profile;

FIG. 4 schematically illustrates the detection of DNA molecules by PCR;

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference. Embodiments of the present invention relate to methods for tracing the flow of liquids with superior sensitivity using nucleic acids of specific sequences as tracers. In one aspect, the invention relates to a significant improvement of the detection sensitivity using nucleic acids of specific sequences as tracers. For example, the present invention provides an improved tracing method whereby even a single DNA molecule in a sample can be detected by an enzymatic amplification method such as PCR. As used herein, the terms “DNA”, a “probe”, a “tracer”, a “nucleic acid”, a “vector”, a “plasmid”, an “enzyme”, a “liquid”, “PCR”, “seepage”, “leakage”, “piping”, and “signal” are to be taken in their broadest context.

In one general aspect, the present invention relates to a method for detecting, locating and monitoring fluid seepage and leakage from a hydraulic work with superior sensitivity. The method comprising: (i) designing a specific DNA sequence with a specific length; (ii) producing and using the nucleic acid containing the DNA sequence as the probe and applying the probe to a proper location of the hydraulic work; (iii) taking samples from specific locations that may contain the probe; (iv) amplifying the probe in the samples by an enzymatic amplification method; and (v) determining the amount or copy number of the probe in the samples to analyze fluid seepage and leakage from the hydraulic work.

In another general aspect, the present invention relates to a method for tracing the flow of the groundwater or underground water with superior sensitivity. The method comprising: (i) designing a specific DNA sequence with a specific length; (ii) producing and using the nucleic acid containing the DNA sequence as the probe and applying the probe to a proper location of the groundwater or underground water; (iii) taking samples from specific locations that may contain the probe; (iv) amplifying the probe in the samples by an enzymatic amplification method; and (v) determining the amount or copy number of the probe in the samples to analyze the flow of the groundwater or underground water.

In a further aspect, the present invention relates to a method for tracing the flow of liquids with superior sensitivity. The method comprising: (i) designing a specific DNA sequence with a specific length; (ii) producing and using the nucleic acid containing the DNA sequence as the probe and applying the probe to a proper location of the liquid body; (iii) taking samples from specific locations that may contain the probe; (iv) amplifying the probe in the samples by an enzymatic amplification method; and (v) determining the amount or copy number of the probe in the samples to analyze the flow of the liquid.

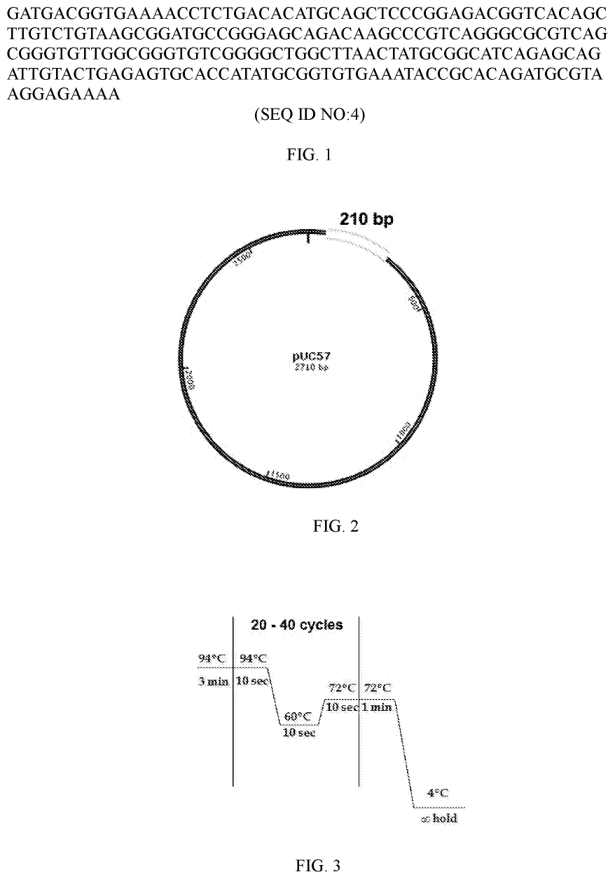

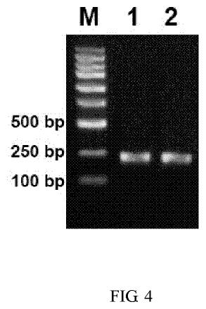

Embodiments of the invention relate to specific DNA sequences with specific lengths as probes or tracers. For example, as illustrated in FIG. 1, a relatively long DNA sequence of 210 base pair (bp) with the sequence specified (SEQ ID NO:4) can be used as a DNA probe or tracer. Contrast to the classical probes or tracers which cannot be amplified, this DNA probe can be amplified by more than 1 millionfold by an enzymatic amplification method and then easily detected. As illustrated in FIG. 4, the specific DNA sequence of the vector was amplified by PCR using a pair of primers, and a clear DNA band can now be seen, which demonstrated the presence of the DNA tracer.

In one embodiment of the invention, the DNA probe or tracer comprises one of the nucleic acids selected from, but not limited to, for example, a single strand DNA, a double strand DNA, a circular single strand DNA, a circular double strand DNA, a plasmid, etc.

It is apparent to those skilled in the art that the present invention includes modifications to the above-mentioned embodiments to further improve the nucleic acid probes or tracers. These modifications include, but are not are limited to, adding one or more chemical groups to the bases of the nucleic acids, adding one or more chemical groups to the ends of the nucleic acids, replacing the phosphate with phosphorothioate, etc. For example, one can replace the oxygen atom of the phosphodiester moiety of the DNA backbone with a sulphur atom, and the resulting modified DNA shows resistance to nucleases and thus has better stability.

It is apparent to those skilled in the art that the DNA sequence to be used as a tracer comprises one of the nucleic acids selected from, but not limited to, for example, a nucleic acid sequence present in Nature, an artificial sequence, a combination of artificial sequences and nucleic acid sequences present in Nature, etc.

It is also apparent to those skilled in the art that the nucleic acid probes can be made by one of the methods selected from, but not limited to, for example, chemical synthesis, PCR amplification of an amplicon, restriction enzyme digestion of nucleic acids, plasmid preparation, etc.

It is also apparent to those skilled in the art that the size of nucleic acid probes can be varied from 20 bp to more than a thousand bp.

According to embodiments illustrated in FIG. 3, a plasmid DNA, which is a double strand circular DNA, can also be used as the probe or tracer.

In the above-mentioned embodiments, those skilled in the art will know that the plasmid can be prepared from cell culture such as bacteria culture at any scale, thus to provide μg to even kg of the DNA probe.

In the above-mentioned embodiments, those skilled in the art will know that the plasmid probe or tracer can be detected by PCR using many possible pairs of primers. In the above-mentioned embodiments, those skilled in the art will know that multiple nucleic acid probes or tracers can be used simultaneously and then detected by PCR using many possible pairs of primers.

In another embodiment of the present invention, the nucleic acid probe or tracer can be amplified by an enzymatic method thus to give high sensitivity. The enzymatic method is selected from the group consisting of, but not limited to, a thermal cycling method, an isothermal method, etc.

In the above-mentioned embodiments, those skilled in the art will know that a thermal cycling method can include, but not limited to, PCR, real-time PCR, multiplex PCR, single-molecule PCR (SM-PCR), touch-down PCR, gradient PCR, etc.

In the above-mentioned embodiments, those skilled in the art will also know that an isothermal method can include, but not limited to, strand displacement amplification, self-sustained sequence replication, rolling circle amplification, loop mediated amplification and helicase dependent amplification, etc.

In the above-mentioned embodiments, those skilled in the art will know that under similar conditions, the amount of DNA from an enzymatic amplification method is proportional to the copy number of the nucleic acid probe in the sample, thus the amount of DNA from the enzymatic amplification can be used to analyze and determine the liquid flow of interest.

In the above-mentioned embodiments, those skilled in the art will also know that the nucleic acid probe in the samples can be captured, enriched or concentrated to further increase the detection sensitivity. The capture or concentration methods include, but not limited to, for example, ethanol precipitation, bead binding, membrane binding, etc.

Various embodiments of the invention have now been described. It is to be noted, however, that this description of these specific embodiments is merely illustrative of the principles underlying the inventive concept. It is therefore contemplated that various modifications of the disclosed embodiments will, without departing from the spirit and scope of the invention, be apparent to persons skilled in the art.

The following specific examples are further illustrative of the nature of the invention, it needs to be understood that the invention is not limited thereto.

### Example

pUC57 plasmid (as illustrated in FIG. 2) was prepared following the standard procedure: E. coli transformed with pUC57 DNA was grown in LB medium and the plasmids were prepared using Qiagen miniprep kit (Qiagen) following manufacturer's directions. The plasmid DNA was eluted with the elution buffer of 10 mM Tris, 1 mM EDTA at pH 8.0, and the DNA concentration was obtained by OD absorption at 260 nm.

FIG. 4 illustrates the detection of DNA molecules by PCR. pUC57 vector was used as the template for PCR amplification using a forward primer (Pf: GGTGATGACGGTGAAAACCTC) (SEQ ID NO:1) and a reverse primer (Pr: TTTCTCCTTACGCATCTGTGC) (SEQ ID NO:2). The 50 μl PCR mixture contained 1 μl of the template DNA (0.5 ng/μl of pUC57), 1 μl of each primer (10 μM), 5 μl of 10×Taq Buffer, 1 μl of Taq DNA Polymerase (2.5 U/μl), 3 μl of MgCl₂ (25 mM), 4 μl of dNTP mixture (2.5 mM of each dNTP) and 34 μl of water. PCR was performed as follows: 1 cycle of denaturation at 94° C. for 5 min, 40 cycles of denaturation at 94° C. for 30 s, annealing at 60° C. for 30 s, and extension at 72° C. for 30 s, followed by 1 cycle of the final extension for 5 min at 72° C. Then, 5 μl of each PCR reaction was mixed with 1 μl of 6× loading buffer and then loaded onto a 2% agarose gel for electrophoresis. A clear band (Lane 1 and 2) of about 210 bp DNA was seen, Lane M is a DNA molecular marker.

The DNA sequence of pUC57 vector:

(SEQ ID NO: 3)
TCGCGCGTTTCGGTGATGACGGTGAAAACCTCTGACACATGCAGCTCC
CGGAGACGGTCACAGCTTGTCTGTAAGCGGATGCCGGGAGCAGACAAG
CCCGTCAGGGCGCGTCAGCGGGTGTTGGCGGGTGTCGGGGCTGGCTTA
ACTATGCGGCATCAGAGCAGATTGTACTGAGAGTGCACCATATGCGGT
GTGAAATACCGCACAGATGCGTAAGGAGAAAATACCGCATCAGGCGCC
ATTCGCCATTCAGGCTGCGCAACTGTTGGGAAGGGCGATCGGTGCGGG
CCTCTTCGCTATTACGCCAGCTGGCGAAAGGGGGATGTGCTGCAAGGC
GATTAAGTTGGGTAACGCCAGGGTTTTCCCAGTCACGACGTTGTAAAA
CGACGGCCAGTGAATTCGAGCTCGGTACCTCGCGAATGCATCTAGATA
TCGGATCCCGGGCCCGTCGACTGCAGAGGCCTGCATGCAAGCTTGGCG
TAATCATGGTCATAGCTGTTTCCTGTGTGAAATTGTTATCCGCTCACA
ATTCCACACAACATACGAGCCGGAAGCATAAAGTGTAAAGCCTGGGGT
GCCTAATGAGTGAGCTAACTCACATTAATTGCGTTGCGCTCACTGCCC
GCTTTCCAGTCGGGAAACCTGTCGTGCCAGCTGCATTAATGAATCGGC
CAACGCGCGGGGAGAGGCGGTTTGCGTATTGGGCGCTCTTCCGCTTCC
TCGCTCACTGACTCGCTGCGCTCGGTCGTTCGGCTGCGGCGAGCGGTA
TCAGCTCACTCAAAGGCGGTAATACGGTTATCCACAGAATCAGGGGAT
AACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCT
GACGAGCATCACAAAAATCGACGCTCAAGTCAGAGGTGGCGAAACCCG
ACAGGACTATAAAGATACCAGGCGTTTCCCCCTGGAAGCTCCCTCGTG
CGCTCTCCTGTTCCGACCCTGCCGCTTACCGGATACCTGTCCGCCTTT
CTCCCTTCGGGAAGCGTGGCGCTTTCTCATAGCTCACGCTGTAGGTAT
CTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTT
GAGTCCAACCCGGTAAGACACGACTTATCGCCACTGGCAGCAGCCACT
GGTAACAGGATTAGCAGAGCGAGGTATGTAGGCGGTGCTACAGAGTTC
TTGAAGTGGTGGCCTAACTACGGCTACACTAGAAGAACAGTATTTGGT
ATCTGCGCTCTGCTGAAGCCAGTTACCTTCGGAAAAAGAGTTGGTAGC
TCTTGATCCGGCAAACAAACCACCGCTGGTAGCGGTGGTTTTTTTGTT
TGCAAGCAGCAGATTACGCGCAGAAAAAAAGGATCTCAAGAAGATCCT
TTGATCTTTTCTACGGGGTCTGACGCTCAGTGGAACGAAAACTCACGT
TAAGGGATTTTGGTCATGAGATTATCAAAAAGGATCTTCACCTAGATC
CTTTTAAATTAAAAATGAAGTTTTAAATCAATCTAAAGTATATATGAG
TAAACTTGGTCTGACAGTTACCAATGCTTAATCAGTGAGGCACCTATC
TCAGCGATCTGTCTATTTCGTTCATCCATAGTTGCCTGACTCCCCGTC
GTGTAGATAACTACGATACGGGAGGGCTTACCATCTGGCCCCAGTGCT
GCAATGATACCGCGAGACCCACGCTCACCGGCTCCAGATTTATCAGCA
ATAAACCAGCCAGCCGGAAGGGCCGAGCGCAGAAGTGGTCCTGCAACT
TTATCCGCCTCCATCCAGTCTATTAATTGTTGCCGGGAAGCTAGAGTA
AGTAGTTCGCCAGTTAATAGTTTGCGCAACGTTGTTGCCATTGCTACA
GGCATCGTGGTGTCACGCTCGTCGTTTGGTATGGCTTCATTCAGCTCC
GGTTCCCAACGATCAAGGCGAGTTACATGATCCCCCATGTTGTGCAAA
AAAGCGGTTAGCTCCTTCGGTCCTCCGATCGTTGTCAGAAGTAAGTTG
GCCGCAGTGTTATCACTCATGGTTATGGCAGCACTGCATAATTCTCTT
ACTGTCATGCCATCCGTAAGATGCTTTTCTGTGACTGGTGAGTACTCA
ACCAAGTCATTCTGAGAATAGTGTATGCGGCGACCGAGTTGCTCTTGC
CCGGCGTCAATACGGGATAATACCGCGCCACATAGCAGAACTTTAAAA
GTGCTCATCATTGGAAAACGTTCTTCGGGGCGAAAACTCTCAAGGATC
TTACCGCTGTTGAGATCCAGTTCGATGTAACCCACTCGTGCACCCAAC
TGATCTTCAGCATCTTTTACTTTCACCAGCGTTTCTGGGTGAGCAAAA
ACAGGAAGGCAAAATGCCGCAAAAAAGGGAATAAGGGCGACACGGAAA
TGTTGAATACTCATACTCTTCCTTTTTCAATATTATTGAAGCATTTAT
CAGGGTTATTGTCTCATGAGCGGATACATATTTGAATGTATTTAGAAA
AATAAACAAATAGGGGTTCCGCGCACATTTCCCCGAAAAGTGCCACCT
GACGTCTAAGAAACCATTATTATCATGACATTAACCTATAAAAATAGG
CGTATCACGAGGCCCTTTCGTC.

1: An integrated method for detecting, locating and monitoring fluid seepage and leakage from a hydraulic work with superior sensitivity, the method comprising using a DNA sequence as a probe, capturing the probe and amplifying the probe by an enzymatic amplification method.

2: A method of detecting, locating and monitoring fluid seepage and leakage from a hydraulic work with superior sensitivity, the method comprising: (i) designing a specific DNA sequence; (ii) using a nucleic acid containing the DNA sequence as a probe and applying the probe to a proper location of the hydraulic work; (iii) taking samples that may contain the probe; (iv) amplifying the probe in the samples by an enzymatic amplification method; and (v) determining an amount or copy number of the probe in the samples to analyze fluid seepage and leakage from the hydraulic work.

3: The method of claim 2, wherein the DNA sequence used as the probe is a sequence present in Nature.

4: The method of claim 2, wherein the DNA sequence used as the probe is an artificial sequence not present in Nature.

5: The method of claim 2, wherein the DNA sequence used as the probe is a combination of natural sequences and artificial sequences.

6: The method of claim 2, wherein the nucleic acid containing the DNA sequence as the probe is double stranded.

7: The method of claim 2, wherein the nucleic acid containing the DNA sequence as the probe is single stranded.

8: The method of claim 2, wherein the nucleic acid containing the DNA sequence as the probe is chemically synthesized.

9: The method of claim 2, wherein the nucleic acid containing the DNA sequence as the probe is chemically modified.

10: The method of claim 2, wherein the nucleic acid containing the DNA sequence as the probe is linear.

11: The method of claim 2, wherein the nucleic acid containing the DNA sequence as the probe is circular.

12: The method of claim 2, wherein the nucleic acid containing the DNA sequence as the probe is made by an enzymatic method.

13: The method of claim 2, wherein the nucleic acid containing the DNA sequence as the probe is originally produced by a host cell, which is selected from the group consisting of a bacteria cell, an yeast cell, an insect cell, a fungal cell, a mammalian cell, and a plant cell.

14: The method of claim 2, wherein the enzymatic amplification method is a thermal cycling method.

15: The method of claim 2, wherein the enzymatic amplification method is an isothermal method.

16: The method of claim 2, wherein multiple DNA sequences are simultaneously used as tracers to trace the fluid seepage and leakage from the hydraulic work.

17: The method of claim 12, wherein the enzymatic method is a PCR process.

18: The method of claim 14, wherein the thermal cycling method is polymerase chain reaction (PCR).

19: The method of claim 15, wherein the isothermal method is isothermal rolling circle amplification or multiple-displacement amplification.


SEQUENCE LISTING (4 sequences)

SEQ ID NO: 1 - DNA, 21 bp, Artificial Sequence
  forward primer
ggtgatgacggtgaaaacctc

SEQ ID NO: 2 - DNA, 21 bp, Artificial Sequence
  reverse primer
tttctccttacgcatctgtgc

SEQ ID NO: 3 - DNA, 2710 bp, Artificial Sequence
  pUC57
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 210 bp, Artificial Sequence
  DNA tracer
gatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaa